(19) United States
(12) Patent Application Publication
(10) Pub. No.: US 2025/0155416 A1
(43) Pub. Date: May 15, 2025

(54) Portable Inflatable Hood With Self-Contained Ballast for Off-Gas Testing of Aeration and Emissions at Wastewater Treatment Facilities

(71) Applicant: Carollo Engineers, Inc., Costa Mesa, CA (US)

(72) Inventors: Samuel Reifsnyder, Concord, CA (US); Greg Stanczak, Boise, ID (US)

(21) Appl. No.: 18/946,282

(22) Filed: Nov. 13, 2024

Related U.S. Application Data
(60) Provisional application No. 63/599,110, filed on Nov. 15, 2023.

(51) Int. Cl. G01N 33/00

(52) U.S. Cl. CPC G01N 33/0009

(57) ABSTRACT

A portable inflatable hood with self-contained ballast for off-gas testing includes a primary structure configured to form a perimeter, a secondary structure surrounding the primary structure, a top element spanning the primary structure and configured to form an enclosure with the primary structure, a sheet configured to connect the primary structure to the secondary structure, a vent, and a flange configured to receive a tube. A method of deploying and using a portable inflatable hood for off-gas testing includes providing a portable inflatable hood, providing an air pump, inflating the portable inflatable hood with the air pump, connecting the portable inflatable hood to an analyzer with a hose or tube, positioning the portable inflatable hood in an area where off-gas is to be collected, securing the portable inflatable hood to a structure outside of the area where off-gas is to be collected, and collecting off-gas with the portable inflatable hood.

### CROSS REFERENCE TO RELATED APPLICATION

This application claims the benefit of U.S. Provisional Patent Application No. 63/599,110, which was filed Nov. 15, 2023, the disclosure of which is hereby incorporated by reference in its entirety.

### BACKGROUND

### 1. Field

The present disclosure relates to a portable inflatable hood device for off-gas testing and, more specifically, to a portable inflatable hood with self-contained ballast for off-gas testing of aeration and emissions at wastewater treatment facilities.

### 2. Technical Considerations

Aeration is a vital and commonly used process used in the treatment of wastewater at most wastewater treatment plants. Aeration is an extremely energy-intensive process, accounting for approximately 45%-75% of the energy consumed during the entire treatment process. There are several types of aeration systems, diffused aeration being the most common. During diffused aeration, air or pure oxygen is injected and dissolved in the wastewater within the biological reactors of a wastewater treatment plant. The amount of dissolved oxygen in the wastewater is thereby increased, and the residual oxygen in the gas phase, along with other gas species, is released from the surface of the aeration tanks at a wastewater treatment plant.

Off-gas testing is the process by which the gases exiting the aerated zones are collected and analyzed. As the gases are released into the air, hoods above the aerated zones collect the gases and transport them to an analyzer to determine their content. The various gas species that are captured and analyzed include oxygen (O₂), carbon dioxide (CO₂), methane (CH₄), and nitrous oxide (N₂O), among others. Measuring these off-gases provides numerous data points for evaluating the performance and efficiency of the diffused aeration system. This information is critical for operations including designing diffuser systems, determining optimal size of blowers, developing an optimal diffuser cleaning schedule, calibrating process models, and assessing existing operating conditions.

Off-gas testing additionally provides data for studying the concentrations and emission rate of greenhouse gases such as methane (CH₄) and nitrous oxide (N₂O) that are emitted by the system. N₂O emissions have the potential to account for as much as 83% of a wastewater treatment plant's operational carbon footprint. Direct greenhouse gas emissions from wastewater treatment plants are difficult to quantify, and quantification is typically accomplished using annual average values known from literature. However, greenhouse gas emissions significantly fluctuate among different wastewater treatment plants and even within the same wastewater treatment plant, with emissions varying across different seasons and throughout a given day. Utilizing a standard emission factor to estimate emissions from a specific wastewater treatment plant has limited usefulness. Accurate emission calculations from a specific wastewater treatment plant require continuous real-time measurements, such as through off-gas testing. Therefore, the ability to monitor and quantify greenhouse gas emissions through off-gas testing plays a critical role for many utilities trying to meet their climate targets.

Although off-gas testing provides valuable information, its widespread use is limited by several factors linked to existing off-gas hoods and their methods of deployment and use. Existing off-gas testing hoods typically include a rigid structure manufactured from a variety of materials including timber, plastic, metal, or a combination thereof. In some instances, off-gas testing hoods are put together from off-the-shelf products such as storage bins which may be framed or weighted with timber or other materials. For an off-gas testing hood to be effective, a certain weight is required to provide adequate ballast, which prevents the hood from lifting during testing. Lifting of the hood during testing risks the entrapment of air in the collected off-gas stream, which impacts the results of the testing and the analysis thereof. This requirement paired with rudimentary fabrication often results in heavy and bulky structures that either need to be pre-assembled and shipped to the wastewater treatment facility ahead of the testing or need to be built on-site in the days preceding the off-gas testing.

If an off-gas hood needs to be shipped ahead of testing, there is increased cost associated with shipping the existing bulky off-gas hoods. Similarly, the bulky nature of existing off-gas hoods requires time for pre-assembly ahead of the off-gas testing, which leads to additional labor time, and thus increased overall costs of the off-gas testing service. The bulky and heavy nature of existing off-gas hoods also typically requires multiple people and/or heavy machinery to lift and move the hood, such as during placement of the hood inside aeration basins at a wastewater treatment facility. This poses safety risks and increased labor costs, as well as increased operational costs associated with the rental of the lifting equipment.

In general, it is advantageous for an off-gas hood to have a large sampling area, as a larger sampling area can collect a greater volume of off-gas, and thus provide a more representative sample and more accurate data. However, the sampling area of existing off-gas hoods is limited due to their bulky and heavy nature as well as their ad-hoc construction. This limitation makes the overall off-gas testing results more susceptible to measurement errors and biases due to preferential off-gassing plumes of other heterogeneous conditions along the basins. For example, a certain area of an aerated zone may give off significantly different off-gas flux rates with varying levels of concentration for oxygen or other gases compared to an immediately adjacent area of the same aerated zone.

In view of the foregoing, there exists a need for a portable inflatable hood with self-contained ballast for off-gas testing of aeration and emissions at wastewater treatment facilities, which allows for easier, faster, safer, and more reliable deployment and use, as well for improved and higher volume testing and decreased overall cost.

### SUMMARY

Accordingly, non-limiting embodiments or aspects of the present disclosure are directed to a portable inflatable hood device for off-gas testing and, more specifically, to a portable inflatable hood with self-contained ballast for off-gas testing of aeration and emissions at wastewater treatment facilities, which allows for easier, faster, safer, and more reliable deployment and use, as well for improved and higher volume testing and decreased cost.

According to non-limiting embodiments or aspects of the disclosure, a portable inflatable hood device for off-gas testing includes a primary structure, a secondary attached to the primary structure, and a top element spanning the primary structure. The primary structure and the top element form an enclosure, and the secondary forms a ballast.

According to non-limiting embodiments or aspects of the disclosure, a system for off-gas testing includes: an inflatable hood having a primary structure and a top element forming an enclosure, and a secondary structure forming a ballast; an analyzer connects to a flange of the inflatable hood; and an air pump.

According to non-limiting embodiments or aspects of the disclosure, a method of using a device for off-gas testing includes providing a portable inflatable hood device, the device including at least one primary structure, at least one secondary structure surrounding the at least one primary structure, a top element spanning the at least one primary structure and configured to form an enclosure with the at least one primary structure, a sheet connected to one of the at least one secondary structure, at least one vent, and at least one flange configured to receive a hose. The method further includes providing an air pump, inflating the portable inflatable hood device with the air pump, connecting the device to an analyzer with a hose, positioning the device in an area where off-gas is to be collected, securing the device to a structure outside of the area where off-gas is to be collected, and collecting off-gas with the device.

Non-limiting illustrative examples of embodiments of the present disclosure will now be described in the following numbered clauses.

Clause 1: A device for off-gas testing, comprising: a primary structure; a secondary structure attached to the primary structure; and a top element spanning the primary structure; wherein the primary structure and the top element form an enclosure, and wherein the secondary structure forms a ballast.

Clause 2: The device of clause 1, wherein the primary structure forms a perimeter of the enclosure.

Clause 3: The device of clause 1 or 2, wherein the secondary structure surrounds the primary structure.

Clause 4: The devices of any of clauses 1-3, wherein the primary structure comprises at least one inflatable tube.

Clause 5: The devices of any of clauses 1-4, wherein the at least one secondary structure comprises at least one hollow tube.

Clause 6: The devices of any of clauses 1-5, wherein the at least one hollow tube is configured to receive a fluid.

Clause 7: The devices of any of clauses 1-6, wherein the at least one hollow tube is at least partially filled with a liquid to form the ballast.

Clause 8: The devices of any of clauses 1-7, further comprising a sheet attaching the at least one hollow tube to the primary structure.

Clause 9: The devices of any of clauses 1-8, wherein the at least one hollow tube, the sheet, and the primary structure define a space, and wherein the space is at least partially filled with a liquid to form the ballast.

Clause 10: The devices of any of clauses 1-9, further comprising a sheet attaching a first hollow tube of the at least one hollow tube to a second hollow tube of the at least one hollow tube.

Clause 11: The devices of any of clauses 1-10, wherein the first hollow tube, the sheet, and the second hollow tube define a space, and wherein the space is at least partially filled with a liquid to form the ballast.

Clause 12: The devices of any of clauses 1-11, further comprising at least one flange configured to receive a hose and form a fluid connection between the enclosure and the hose.

Clause 13: The devices of any of clauses 1-12, further comprising at least one additional flange comprising a fitting for connecting one or more sensors.

Clause 14: The devices of any of clauses 1-13, further comprising at least one vent configured to release one or more fluid from the enclosure.

Clause 15: The devices of any of clauses 1-14, further comprising at least one fastener, wherein the at least one fastener comprises one of a hook, a loop, an eyelet, or a handle.

Clause 16: A system for off-gas testing, comprising: an inflatable hood comprised of: a primary structure and a top element forming an enclosure; and a secondary structure forming a ballast; and an analyzer connected to a flange of the inflatable hood; and an air pump.

Clause 17: The system of clause 16, further comprising: a hose fluidly connecting the analyzer to the flange and the enclosure, wherein the inflatable hood further comprises a vent, a fastener, and at least one sensor.

Clause 18: The system of clause 16 or 17, wherein at least a portion of the system is provided in a case.

Clause 19: A method of using a device for off-gas testing, comprising: providing a portable inflatable hood device comprising: at least one primary structure configured to form a perimeter; at least one secondary structure surrounding the at least one primary structure; a top element spanning the at least one primary structure and configured to form an enclosure with the at least one primary structure; a sheet connected to one of the at least one secondary structure; at least one vent; and at least one flange configured to receive a hose, providing an air pump, inflating the portable inflatable hood with the air pump, connecting the device to an analyzer with a hose, positioning the device in an area where off-gas is to be collected, securing the device to a structure outside of the area where off-gas is to be collected, and collecting off-gas with the portable inflatable hood device.

Clause 20: The method of clause 19, further comprising: detecting one or more properties of the collected off-gas with the analyzer, and analyzing data comprised of the one or more properties detected by the analyzer.

Further details and advantages of the various examples described in detail herein will become clear upon reviewing the following detailed description of the various examples in conjunction with the accompanying drawing figures.

### BRIEF DESCRIPTION OF THE DRAWINGS

Additional advantages and details are explained in greater detail below with reference to the exemplary non-limiting embodiments or aspects that are illustrated in the accompanying schematic figures, in which:

FIG. 1 is a perspective view of a non-limiting embodiment or aspect of a portable inflatable hood device for off-gas testing in accordance with the present disclosure;

FIG. 2 is a plan view of a non-limiting embodiment or aspect of a portable inflatable hood device for off-gas testing in accordance with the present disclosure;

FIG. 3 is a sectional view of a non-limiting embodiment or aspect of a portable inflatable hood device for off-gas testing in accordance with the present disclosure;

FIG. 4 is a sectional view of a non-limiting embodiment or aspect of a portable inflatable hood device for off-gas testing in accordance with the present disclosure;

FIG. 5 is a sectional view of a non-limiting embodiment or aspect of a portable inflatable hood device for off-gas testing in accordance with the present disclosure;

FIG. 6 is a sectional view of a non-limiting embodiment or aspect of a portable inflatable hood device for off-gas testing in accordance with the present disclosure;

FIG. 7 is a sectional view of a non-limiting embodiment or aspect of a portable inflatable hood device for off-gas testing in accordance with the present disclosure;

FIG. 8 is a perspective view of a non-limiting embodiment or aspect of a portable inflatable hood device for off-gas testing in accordance with the present disclosure;

FIG. 9 is a perspective view of a non-limiting embodiment or aspect of a portable inflatable hood device for off-gas testing in accordance with the present disclosure; and

FIGS. 10A and 10B are a perspective view and a side view of a non-limiting embodiment or aspect of a portable inflatable hood device for off-gas testing in accordance with the present disclosure.

Corresponding reference characters indicate corresponding parts throughout the several views. The exemplifications set out herein illustrate exemplary non-limiting embodiments or aspects of the disclosure, and such embodiment or aspects are not to be construed as limiting the scope of the disclosure in any manner.

### DETAILED DESCRIPTION

For purposes of the description hereinafter, the terms “end”, “upper”, “lower”, “right”, “left”, “vertical”, “horizontal”, “top”, “bottom”, “lateral”, “longitudinal”, and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one”. As used in the specification and the claims, the singular form of “a”, “an”, and “the” include plural referents, such as unless the context clearly dictates otherwise. Where only one item is intended, the term “one” or similar language is used. Also, as used herein, the terms “has”, “have”, “having”, or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise.

Non-limiting embodiments or aspects of the present disclosure are directed to a portable inflatable hood device with self-contained ballast for off-gas testing of aeration and emissions at wastewater treatment facilities (“off-gas hood”). According to non-limiting embodiments or aspect of the present disclosure, the device may include a primary structure, a secondary structure attached to the primary structure, and a top element spanning the at least one primary structure. The primary structure and the top element may form an enclosure and the secondary structure may form a ballast.

In some non-limiting embodiments or aspects, the primary structure may form a perimeter of the enclosure. In some non-limiting embodiments or aspects, the secondary structure may surround the primary structure. In some non-limiting embodiments or aspects, the primary structure may include at least one inflatable tube. In some non-limiting embodiments or aspects, the secondary structure may include at least one hollow tube. In some non-limiting embodiments or aspects, the at least one hollow tube may be configured to receive a fluid. In some non-limiting embodiments or aspects, the at least one hollow tube may be further configured to receive a ballast material.

In some non-limiting embodiments or aspects, the device may further include a sheet attaching the at least one hollow tube to the primary structure. In some non-limiting embodiments or aspects, the at least one hollow tube, the sheet, and the primary structure may define a space, and the space may be at least partially filled with a ballast material.

In some non-limiting embodiments or aspects, the device may further include a sheet attaching a first hollow tube of the at least one hollow tube to a second hollow tube of the at least one hollow tube. In some non-limiting embodiments or aspects, the first hollow tube, the sheet, and the second hollow tube may define a space, and the space may be at least partially filled with a ballast material.

In some non-limiting embodiments or aspects, the device may further include at least one flange configured to receive a hose and form a fluid connection between the enclosure and the hose. In some non-limiting embodiments or aspects, the device may further include at least one additional flange comprising a fitting for connecting one or more sensors. In some non-limiting embodiments or aspects, the device may further include at least one vent configured to release one or more fluid from the enclosure. In some non-limiting embodiments or aspects, the device may further include at least one fastener, and the at least one fastener may be one of a hook, a loop, an eyelet, or a handle.

In this way, non-limiting embodiments or aspects of the present disclosure allow for a portable inflatable hood device with self-contained ballast for off-gas testing of aeration and emissions at wastewater treatment facilities, which allows for easier, faster, safer, and more reliable deployment and use, as well for improved and higher volume testing and decreased cost.

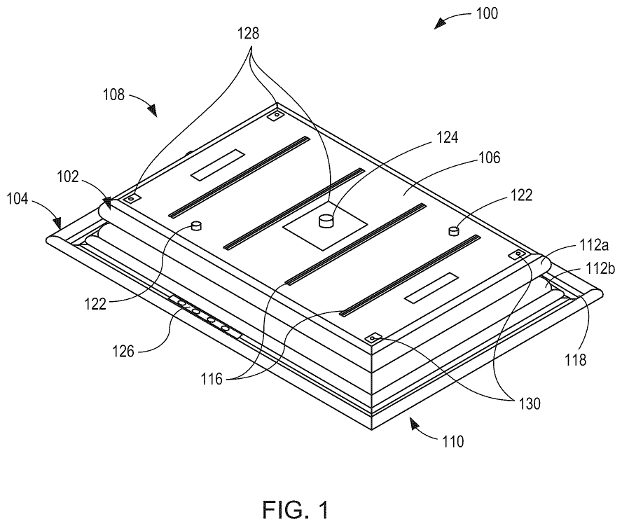

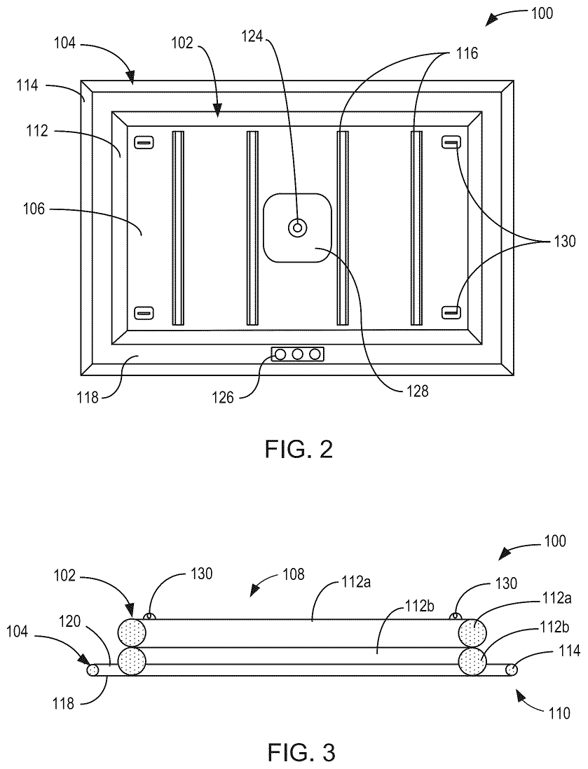

Referring now to FIGS. 1-3, FIG. 1 is a perspective view, FIG. 2 is a plan view, and FIG. 3 is a sectional view of a non-limiting embodiment or aspect of off-gas hood device 100 in accordance with the present disclosure. In non-limiting embodiments or aspects, as shown in FIG. 1, device 100 may include primary structure 102, secondary structure 104, and top element 106. Primary structure 102 and top element 106 may form an enclosure 108, and secondary structure 104 may form a ballast 110.

In some non-limiting embodiments or aspects, primary structure 102 may form a perimeter of enclosure 108 such that primary structure 102 and top element 106 forming enclosure 108 may define a hood portion of off-gas hood device. In some non-limiting embodiments, secondary structure 104 forming ballast 110 may surround primary structure 102, and thus, may also surround enclosure 108. However, it is contemplated that in some non-limiting embodiments or aspects the primary structure may surround the secondary structure such that the ballast is formed on the interior of the enclosure (i.e., the ballast may be inside the hood).

As shown in FIGS. 1-3, according to some non-limiting embodiments or aspects, the main structure and general shape of device 100 may be square or rectangular. In some non-limiting embodiments or aspects, the main structure and general shape of device 100 may be triangular, circular, semi-circular, octagonal, or any other suitable shape.

With continued references to FIGS. 1-3, in some non-limiting embodiments or aspects, primary structure 102 may include at least one inflatable tube or a plurality of inflatable tubes, such as inflatable tube 112a and/or inflatable tube 112b (collectively, inflatable tubes 112). Inflatable tubes 112 may be hollow cylinders, prims or other like structures. In some non-limiting embodiments or aspects, inflatable tubes 112 may be stacked, layered, or otherwise attached to each other to form primary structure 102 and enclosure 108. As shown in the exemplary non-limiting embodiment or aspect, primary structure 102 may include two inflatable tubes 112a, 112b, which are stacked to form the perimeter of enclosure 108. In some non-limiting embodiments or aspects, inflatable tubes 112 may be inflatable such that inflatable tubes 112 may be at least partially filled or inflated with a fluid, particularly with a gas such as air, to form a semi-rigid structure capable of holding their shape once inflated.

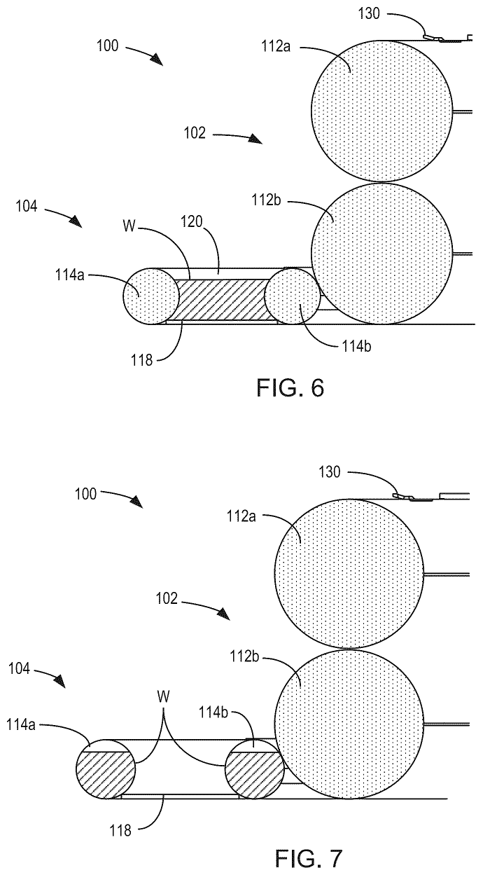

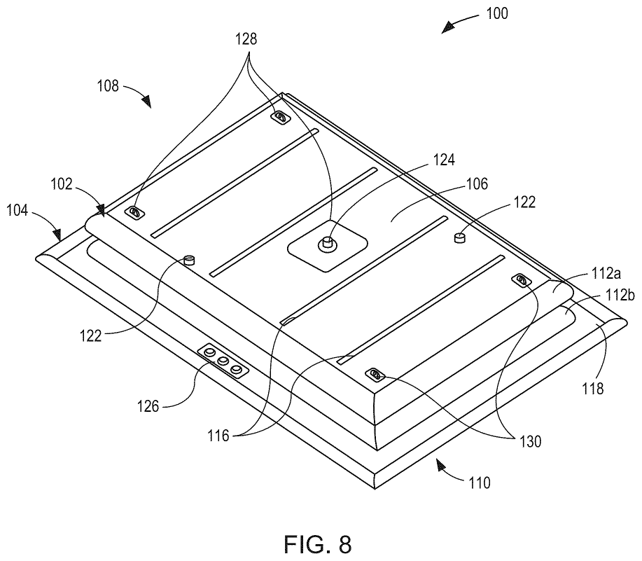

In some non-limiting embodiments or aspects, secondary structure 104 may include at least one hollow tube 114 or a plurality of hollow tubes (e.g., hollow tube 114a and hollow tube 114b as shown in FIGS. 6 and 7), which may be configured to receive a fluid. In some non-limiting embodiments or aspects, at least one hollow tube 114 of secondary structure 104 may be in fluid connection with at least one inflatable tube 112 of primary structure 102. Accordingly, at least one inflatable tube 112 of primary structure 102 and at least one hollow tube of secondary structure 104 may be inflatable and deflatable at the same time and through a single valve. Conversely, at least one inflatable tube 112 of primary structure 102 and at least one hollow tube 114 of secondary structure 104 may not be in fluid connection and may each include a valve for independent inflation and deflation. In some non-limiting embodiments or aspects, multiple hollow tubes 114 may be stacked or otherwise attached to each other such that more than one hollow tube 114 may surround enclosure 108 (e.g., as shown in FIGS. 6 and 7).

As shown in FIGS. 1-3, in some non-limiting embodiments or aspects, enclosure 108 (e.g., the “hood” portion of device 100), may form an interior space or volume which may contain and collect the off-gas for testing. In some non-limiting embodiments or aspects, off-gas hood 100 may include a sprayer inside enclosure 108 with which to spray down the interior surfaces of enclosure 108 to keep it free from buildup of foam and other debris. Such a sprayer may be particularly advantageous for applications in which off-gas hood 100 is deployed for long periods of time for long term testing.

In some non-limiting embodiments or aspects, as shown in FIGS. 1-3, top element 106 of off-gas hood device 100 may include sleeves 116, which may be narrow, elongated sleeves extending along a width or length of top element 106. Sleeves 116 may be configured to accept rods to provide rigidity to top element 106 and to prevent top element 106 from sagging or drooping.

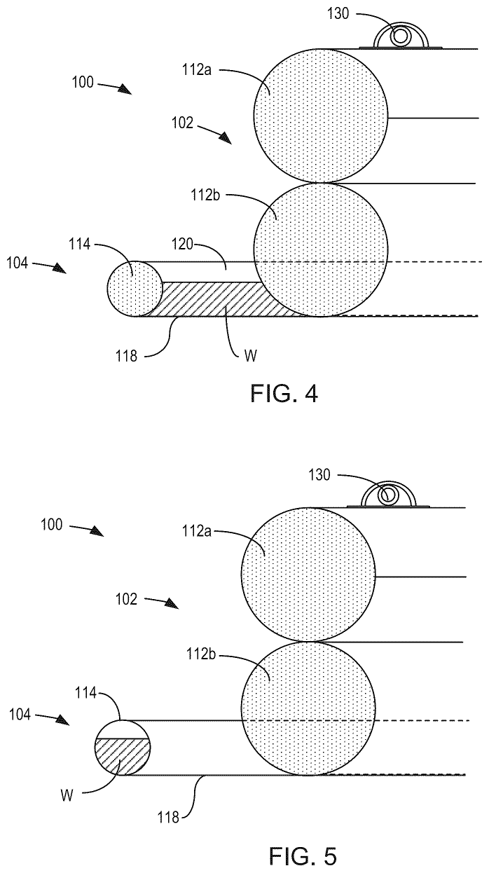

As shown in FIGS. 1-3, in some non-limiting embodiments or aspects, the connection between the primary structure 102 and secondary structure 104 may be achieved through a flat material such as sheet 118. Accordingly, sheet 118 may be configured to connect one of the at least one hollow tube 114 to primary structure 102, such as to an inflatable tube 112 of primary structure 102. In some non-limiting embodiments or aspects, sheet 118 may be configured to connect first hollow tube 114a of secondary structure 104 to a second hollow tube 114b (e.g., as shown in FIGS. 6 and 7). As further discussed below with reference to FIGS. 4-7, sheet 118 may also be configured to ballast off-gas hood device 100 such as by receiving a ballast material and/or by forming a part of the ballast.

As further shown in FIG. 1, in some non-limiting embodiments or aspects, off-gas hood device 100 may include at least one vent 122 configured to release one or more fluid from enclosure 108. Vent 122 may be a one-way breather vent installed in top element 106 and may be configured to maintain consistent pressure inside the enclosure of off-gas hood 100 by allowing air to flow through.

Additionally, as shown in FIGS. 1-3, in some non-limiting embodiments or aspects, off-gas hood device 100 may include at least one flange 124, which may be configured to receive a hose, a conduit or another like structure. In some non-limiting embodiments or aspects, flange 124 may be configured to form a fluid connection between enclosure 108 and the hose. In some non-limiting embodiments or aspects, flange 124 may be installed in top element 106 of device 100 for a quick connection of the hose or conduit transferring the off-gas to an analyzer. In some non-limiting embodiments or aspects, device 100 may include at least one additional flange such as flat flange 126. For example, the additional flange such as flat flange 126 may include connection fittings or screw head ports and may be installed on the flat material (e.g., sheet 118) between primary structure 102 and secondary structure 104. The additional flange(s) may be configured to fasten any number of sensors including liquid-phase sensors such as dissolved oxygen (DO) sensors, nitrogen (NHₓ, NOₓ, N₂O) sensors, total suspended solids (TSS) sensors, pH sensors, oxidation reduction potential (ORP) sensors, and/or any other immersible sensing device.

In some non-limiting embodiments or aspects, device 100 may include at least one fastener 130. Fastener 130 may be a hook, a loop, an eyelet, a handle, or another fastener that may allow a use to move and manipulate the positioning of device 100. Similarly, fastener 130 may be used to anchor or secure device 100 once it is deployed in an area where off-gas testing is to be conducted. In some non-limiting embodiments or aspects, multiple fasteners 130 may be positioned on the surface of top element 106. In other embodiments, fasteners may be positioned anywhere on primary structure 102, secondary structure 104, or anywhere else on device 100 advantageous for manipulating and/or securing device 100.

In some non-limiting embodiments or aspects, reinforcement patches 128 may be provided on off-gas hood device 100 at points subject to stress, which may prevent tearing of the material. Such points where reinforcement patches 128 may be provided include around vents 122, flange 124, and flat flange 126. Additionally, reinforcement patches 128 may be provided at other points of high stress such as around hooks, handles, and eyelets which may be provided for easier maneuverability and stabilization of off-gas hood device 100.

Because off-gas hood device 100 may be comprised of primarily inflatable and flat structures, the weight and bulk is significantly decreased as compared with traditional off-gas hoods constructed of wood, metal, and the like. Moreover, off-gas hood 100 is portable and can be easily moved, packed, and shipped. It is considered that the entirety of off-gas hood device 100 may be contained in a relatively small container such as a hard case or container for transportation and storage. Accordingly, the costs of pre-shipping or pre-assembly of off-gas hood device 100 is greatly reduced compared to existing hoods.

In some non-limiting embodiments or aspects, an inflatable off-gas hood device may be provided as or in a system for off-gas testing. Such a system may include an inflatable hood device 100 including primary structure 102 and top element 106 forming enclosure 108, and secondary structure 104 forming a ballast. The system may further include an analyzer connected to flange 124 of the inflatable hood, and an air pump. In some non-limiting embodiments, the system may also include a hose fluidly connecting the analyzer to flange 124 and enclosure 108. In some non-limiting embodiments or aspects, the inflatable hood may further include vent 122, fastener 130, and at least one sensor. A portion of the system or the entirety of the system may be provided in a case or container for transportation and storage.

The reduced weight of off-gas hood device 100 allows for quick and easy installation by any trained professional, regardless of one's ability to lift heavy objects, physical health, or physical condition. Heavy lifting machinery or equipment, such as a crane, is unnecessary due to the relative light weight of device 100. Further, no support from local staff is required to build, lift, or schedule lifting operations as a single person can inflate, deploy, and secure off-gas hood device 100 during testing. Accordingly, labor hours are saved and cost is reduced.

Further, the portability and reduced weight of inflatable off-gas hood device 100 allows achievement of a larger sized hood, with a larger surface area and volume of the enclosure used to collect the off-gas. In general, the larger the off-gas hood enclosure area is, the greater the amount of off-gas collected, and thus, a larger and more representative sample can be obtained. Off-gas testing using smaller hoods may be more susceptible to measurement errors and biases due to preferential off-gassing plumes of other heterogenous conditions along the basins.

Referring now to FIGS. 4 and 5, FIGS. 4 and 5 are sectional views of non-limiting embodiments or aspects of off-gas hood device 100 in accordance with the present disclosure. As shown in FIGS. 4 and 5, in some non-limiting embodiments or aspects, primary structure 102 may include multiple inflatable tubes 112a, 112b which may be stacked on top of one another. As also shown in FIGS. 4 and 5, secondary structure 104 of device 100 may include one hollow tube 114 attached with sheet 118 to inflatable tube 112b such that secondary structure 104 may surround primary structure 102. As shown in FIG. 4, hollow tube 114, sheet 118, and inflatable tube 112b of primary structure 102 may define space 120. Space 120 may be at least partially filled with a ballast material, such as a fluid, a liquid such as water W, or sand, to form the ballast. Accordingly, device 100 may be ballasted by filling the volume of space 120 above sheet 118 between secondary structure 104 and primary structure 102 with a ballast material such as water W. In some non-limiting embodiments or aspects, such as shown in FIG. 5, the at least one hollow tube 114 may itself be at least partially filled with water W, another fluid or liquid, or any other suitable ballast material, to form the ballast and ballast off-gas hood device 100.

Referring now to FIGS. 6 and 7, FIGS. 6 and 7 are sectional views of non-limiting embodiments or aspects of off-gas hood device 100 in accordance with the present disclosure. As shown in FIGS. 6 and 7, in some non-limiting embodiments or aspects, primary structure 102 may include multiple inflatable tubes 112a, 112b which may be stacked on top of one another. As also shown in FIGS. 6 and 7, secondary structure 104 may include multiple hollow tubes 114a, 114b, which may be attached to each other such that secondary structure 104 may surround primary structure 102. Further, sheet 118 may be configured to connect a first hollow tube such as hollow tube 114a to a second hollow tube such as hollow tube 114b. In some non-limiting embodiments, first hollow tube 114a, sheet 118, and second hollow tube 114b may define space 120.

As shown in FIG. 6, in some non-limiting embodiments or aspects, off-gas hood device 100 may be ballasted by at least partially filling the volume of space 120 above sheet 118 between first hollow tube 114a and second hollow tube 114b with a ballast material. In some non-limiting embodiments or aspects, such as shown in FIG. 7, one of the at least one hollow tube 114 may itself be filled with water W or another ballast material to ballast off-gas hood device 100. In some non-limiting embodiments or aspects, as shown in FIG. 7, multiple hollow tubes 114a, 114b may be at least partially filled with a ballast material such as water W.

In some non-limiting embodiments or aspects, the ballast material used to ballast device 100 may be water W as shown in FIGS. 4-7 or may be any other liquid or solid material (e.g., sand) capable of being added to the space 120 between secondary structure 104 and primary structure 102 or within one or more hollow tube 114 of secondary structure 104 itself. The space 120 may be configured such that it allows sufficient ballast material to be added to adequately ballast (weigh down) off-gas hood device 100.

The addition of ballast material prevents off-gas hood device 100 from lifting and thus risking the entrapment of air in the collected off-gas stream. Water from an access point at the facility may be used to either i) fill space 120 between primary inflatable structure 102 and secondary inflatable structure 104 (or between two hollow tubes 114 of secondary inflatable structure 104), or ii) fill the hollow tubes 114 of secondary inflatable structure 104 themselves. Either volume may be filled with a hose, a tube, or the like, or the volume may be automatically filled due to turbulent movement of the water in which the off-gas hood is deployed. The filling may also be controlled to ensure there is an adequate weight to off-gas hood dimension to ensure optimal ballasting. Therefore, the portability of off-gas hood device 100 is guaranteed, while also ensuring that a representative off-gas sample is collected. However, it is also considered that another traditional ballast material such as timber, sand, metal or the like may instead or additionally be placed on sheet 118 to ballast off-gas hood device 100.

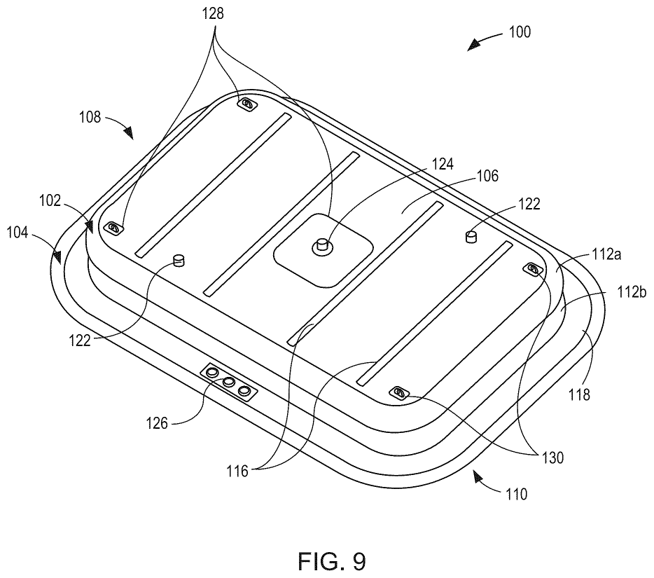

Referring now to FIGS. 8 and 9, FIGS. 8 and 9 are perspective views of non-limiting embodiments or aspects of off-gas hood device 100 in accordance with the present disclosure. In some non-limiting embodiments, as shown in FIG. 8, off-gas hood device 100 may include square corners. Accordingly, both primary structure 102 and secondary structure 104 may include square corners such that the main hood structure, enclosure 108, and the ballasts surrounding the perimeter may be rectangular or square. In some non-limiting embodiments, as shown in FIG. 9, off-gas hood device 100 may include rounded corners. Accordingly, both primary structure 102 and secondary structure 104 may include rounded corners such that the main hood structure, enclosure 108, and the ballasts surrounding the perimeter do not include any square or sharp edges.

In some non-limiting embodiments or aspects, primary structure 102 and secondary structure 104 may include a combination of square and rounded corners such that the main hood structure, enclosure 108, and the ballasts surrounding the perimeter may also include a combination of square and rounded corners, or device 100 may include different shaped corners for each respective structure. Further, primary structure 102 and secondary structure 104 may not include any corners such that the main hood structure enclosure 108, and the ballasts surrounding the perimeter are circular, semi-circular, oval, or other suitable shapes.

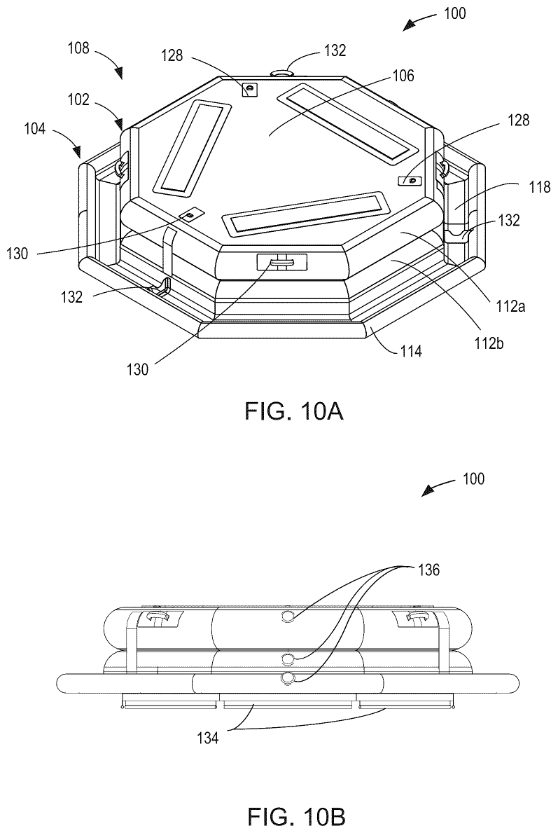

Referring now to FIGS. 10A and 10B, FIGS. 10A and 10B are a perspective view and a side view, respectively, of a non-limiting embodiment or aspect of off-gas hood device 100 in accordance with the present disclosure. In some non-limiting embodiments, as shown in FIGS. 10A and 10B, off-gas hood device 100 may include eight sides. Accordingly, both primary structure 102 and secondary structure 104 may include eight sides. In some non-limiting embodiments or aspects, the eight sides may be equal such that the main hood structure, enclosure 108, and the ballasts surrounding the perimeter may be octagonal.

With continued reference to FIGS. 10A and 10B, in some non-limiting embodiments or aspects, off-gas hood device 100 may further include one or more rib 132 between the primary structure 102 and secondary structure 104 such that the connection between primary structure 102 and 104 may be strengthened by one or more rib 132. In some non-limiting embodiments or aspects, one or more rib 132 may be further connected to sheet 118. Accordingly, one or more rib 132 may be configured to connect one of the at least one hollow tube 114 to primary structure 102, such as to an inflatable tube 112 of primary structure 102. In some non-limiting embodiments or aspects, one or more rib 132 may be configured to connect first hollow tube 114a of secondary structure 104 to a second hollow tube 114b.

In some non-limiting embodiments or aspects, as shown in FIG. 10B, off-gas hood device 100 may further include one or more projections 134 extending downward from any of inflatable tubes 112 of primary structure 102, hollow tubes 114 of secondary structure 104 and sheet 118. In some non-limiting embodiments, or aspects, projections 134 may help to stabilize off-gas hood device 100 and/or prevent movement of off-gas hood device 100 where off-gas hood device 100 is positioned in an area where off-gas is to be collected.

In some non-limiting embodiments or aspects, as shown in FIG. 10B, off-gas hood device 100 may further include one or more port 136 for inflation and deflation of inflatable tubes 112 of primary structure 102, and/or hollow tubes 114 of secondary structure 104. In some non-limiting embodiments or aspects, at least one inflatable tube 112 of primary structure 102 and at least one hollow tube 114 of secondary structure 104 includes port 136 for independent inflation and deflation of primary structure 102 and secondary structure 104. In some non-limiting embodiments, or aspects, each individual inflatable tube 112 and each hollow tube 114 may include a dedicated port 136 for independent inflation and deflation. In some non-limiting embodiments, off-gas hood device 100 may include exactly one port 136 for inflation and deflation of all inflatable tubes 112 and all hollow tubes 114. In some non-limiting embodiments, or aspects, port 136 may be, for example, a valve such as an inflation valve.

Off-gas hood device 100 as discussed herein, including the components thereof (i.e., primary structure 102, secondary structure 104, top element 106, sheet 118, etc.), may be constructed of any suitable material, preferably a waterproof polymer, such as rubber, polyvinyl chloride (PVC), nylon, or combinations thereof.

The present disclosure is also directed to a method of deploying and using a portable inflatable hood device for off-gas testing. According to non-limiting embodiments or aspects of the present disclosure, the method may include providing a portable inflatable hood. In some non-limiting embodiments or aspects, the portable inflatable hood may be the same or substantially similar to off-gas hood device 100 discussed hereinabove with reference to FIGS. 1-9. The portable inflatable hood may include at least one primary structure, at least one secondary structure surrounding the at least one primary structure, and a top element spanning the at least one primary structure and configured to form an enclosure with the at least one primary structure. The portable inflatable hood may further include a sheet connected to one of the at least one secondary structure, at least one vent, and at least one flange configured to receive a hose. In some non-limiting embodiments, the portable inflatable hood may be provided in a container such as a hard case.

In non-limiting embodiments or aspects, the method may further include providing an air pump and inflating the portable inflatable hood with the air pump. The air pump may be a hand pump or an electric pump and may be provided in the same or a different container or hard case as the off-gas hood. Further, the method may include connecting the portable inflatable hood to an analyzer with a hose or conduit. The analyzer may be configured to collect and analyze off-gas via various sensors, readers, and the like. In some non-limiting embodiments or aspects, the analyzer may be provided in the same or a different container or hard case as the off-gas hood and/or the air pump.

Additionally, in non-limiting embodiments or aspects, the method may include positioning the portable inflatable hood in an area where off-gas is to be collected. This area may be an aerated zone of a basin of a wastewater treatment facility. Due to the decreased weight and bulk, positioning the off-gas hood may be carried out by one person. The method may also include securing the portable inflatable hood to a structure outside of the area where off-gas is to be collected. The portable inflatable hood may be secured to a structure outside of the area where off-gas is to be collected with a rope, cable, or the like tied to a hook, handle, eyelet, handle or the like of the portable inflatable hood. In non-limiting embodiments or aspects, the method may further include collecting off-gas with the portable inflatable hood.

In some non-limiting embodiments or aspects, the method may further include detecting one or more properties of the collected off-gas with the analyzer, and analyzing data comprised of the one or more properties detected by the analyzer.

Although the method of deploying and using a portable inflatable hood device for off-gas testing is described herein with reference to off-gas testing at a wastewater treatment facility, it is to be understood that the method is applicable for deployment and use of a portable inflatable hood in other applications such as for other wastewater treatment methods, in other facilities, and for other types of gas testing.

While several examples of the portable inflatable hood device for off-gas testing and methods for deploying and using the same are shown in the accompanying figures and described in detail hereinabove, other aspects will be apparent to and readily made by those skilled in the art without departing from the scope and spirit of the disclosure. Accordingly, the foregoing description is intended to be illustrative rather than restrictive. The invention described hereinabove is defined by the appended claims and all changes to the invention that fall within the meaning and range of equivalency of the claims are to be embraced within their scope.

What is claimed is:

1. A device for off-gas testing, comprising:
a primary structure;
a secondary structure attached to the primary structure; and
a top element spanning the primary structure;
wherein the primary structure and the top element form an enclosure, and
wherein the secondary structure forms a ballast.

2. The device of claim 1, wherein the primary structure forms a perimeter of the enclosure.

3. The device of claim 1, wherein the secondary structure surrounds the primary structure.

4. The device of claim 1, wherein the primary structure comprises at least one inflatable tube.

5. The device of claim 1, wherein the at least one secondary structure comprises at least one hollow tube.

6. The device of claim 5, wherein the at least one hollow tube is configured to receive a fluid.

7. The device of claim 6, wherein the at least one hollow tube is further configured to receive a ballast material.

8. The device of claim 6, further comprising a sheet attaching the at least one hollow tube to the primary structure.

9. The device of claim 8, wherein the at least one hollow tube, the sheet, and the primary structure define a space, and
wherein the space is at least partially filled with a ballast material.

10. The device of claim 6, further comprising a sheet attaching a first hollow tube of the at least one hollow tube to a second hollow tube of the at least one hollow tube.

11. The device of claim 10, wherein the first hollow tube, the sheet, and the second hollow tube define a space, and
wherein the space is at least partially filled with a ballast material.

12. The device of claim 1, further comprising at least one flange configured to receive a hose and form a fluid connection between the enclosure and the hose.

13. The device of claim 12, further comprising at least one additional flange comprising a fitting for connecting one or more sensors.

14. The device of claim 1, further comprising at least one vent configured to release one or more fluid from the enclosure.

15. The device of claim 1, further comprising at least one fastener,
wherein the at least one fastener comprises one of a hook, a loop, an eyelet, or a handle.

16. A system for off-gas testing, comprising:
an inflatable hood comprised of:
a primary structure and a top element forming an enclosure; and
a secondary structure forming a ballast; and
an analyzer connected to a flange of the inflatable hood; and
an air pump.

17. The system of claim 16, further comprising:
a hose fluidly connecting the analyzer to the flange and the enclosure,
wherein the inflatable hood further comprises a vent, a fastener, and at least one sensor.

18. The system of claim 16, wherein at least a portion of the system is provided in a case.

19. A method of using a device for off-gas testing, comprising:
providing a portable inflatable hood, the portable inflatable hood comprising:
at least one primary structure;
at least one secondary structure surrounding the at least one primary structure;
a top element spanning the at least one primary structure and configured to form an enclosure with the at least one primary structure;
a sheet connected to one of the at least one secondary structure;
at least one vent; and
at least one flange configured to receive a hose,
providing an air pump,
inflating the portable inflatable hood with the air pump,
connecting the portable inflatable hood to an analyzer with a hose,
positioning the portable inflatable hood in an area where off-gas is to be collected,
securing the portable inflatable hood to a structure outside of the area where off-gas is to be collected, and
collecting off-gas with the portable inflatable hood.

20. The method of claim 19, further comprising:
detecting one or more properties of the collected off-gas with the analyzer, and
analyzing data comprised of the one or more properties detected by the analyzer.
